(19) United States
(12) Patent Application Publication
(10) Pub. No.: US 2024/0285702 A1
(43) Pub. Date: Aug. 29, 2024

(54) Compositions comprising a cannabinoid and uses thereof

(71) Applicant: Cannabotech Ltd., Herzliya (IL)

(72) Inventors: Galia TEMTSIN KRAYZ, Ashdod (IL); Gadi KLARSFELD, Hod Hasharon (IL); Basem FARES, Horfesh (IL); Dobroslav MELAMED, Rishon Lezion (IL); Pavel KAZHDAN, Sderot (IL)

(21) Appl. No.: 18/565,572

(22) PCT Filed: May 29, 2022
(86) PCT No.: PCT/IL2022/050569

§ 371 Date: Nov. 30, 2023

Related U.S. Application Data
(60) Provisional application No. 63/202,194, filed on May 31, 2021.

(51) Int. Cl. A61K 36/074; A61K 9/00; A61K 9/20; A61K 9/28; A61K 31/00; A61K 45/06

(52) U.S. Cl. CPC A61K 36/074; A61K 9/0053; A61K 9/2031; A61K 9/2813; A61K 9/2826; A61K 9/2866; A61K 9/288; A61K 31/658; A61K 36/071; A61K 45/06; A61K 2236/331; A61K 2236/37

(57) ABSTRACT

The present invention provides pharmaceutical and nutraceutical compositions comprising a cannabinoid, e.g., cannabidiol (CBD), and D-α-tocopherol polyethylene glycol 1000 succinate (TPGS); as well as such nutraceutical compositions which further comprise at least one additional active agent such as a biomass of a higher Basidiomycetes mushroom or a combination thereof, or an extract obtained therefrom.

### TECHNICAL FIELD

The present invention provides pharmaceutical and nutraceutical compositions comprising a cannabinoid, e.g., cannabidiol (CBD), and D-α-tocopheryl polyethylene glycol 1000 succinate (TPGS); as well as such nutraceutical compositions which further comprise an additional active agent such as a biomass of a higher Basidiomycetes mushroom or a combination thereof, or an extract obtained therefrom.

### BACKGROUND ART

The importance of tablet dissolution and drug (biologically active agent) release has been realized by pharmaceutical scientists many decades ago.

Drug absorption from a solid dosage form, after oral administration, depends on the release of the active agent from the product administered; the dissolution or solubilization of said active agent under physiological conditions; and the permeability across the gastrointestinal tract.

Based on this general consideration, the Biopharmaceutics Classification System (BCS) has been developed to provide a scientific approach for prediction of in-vivo pharmacokinetics of immediate release of oral products (Amidon et al., 1995). Specifically, Class 1 includes high solubility—high permeability drugs; Class 2 includes low solubility—high permeability drugs; Class 3 includes high solubility—low permeability drugs; and Class 4 includes low solubility—low permeability drugs. This classification is based on the solubility of a drug which depends on its dose and intestinal permeability in combination with dissolution properties. A drug is considered to be highly permeable when the dose is completely (more specifically, >90%) absorbed; and as highly soluble when the highest clinical dose can be dissolved in 250 ml buffer within a pH range of 1-7.5.

The BCS provides clear rules in determining the rate limiting factors in the gastrointestinal drug absorption process that may be used for selection of drug candidates for development, choice of formulation principle, prediction and elucidation of food interactions, and/or establishing in vitro/in vivo correlations from dissolution testing of solid formulations (Fleisher et al., 1999; Lennernaäs 1998; Lennernäs and Abrahamsson 2005; Polli et al., 2004; Yu et al., 2002; Dressman and Reppas, 2000).

The dissolution of a drug is influenced by several factors such as physicochemical properties (e.g., solubility, crystalline forms, particle size, molecular structure, and diffusivity in the dissolution medium), formulation characteristics (e.g., additives, coatings, and manufacturing parameters), and dissolution method (e.g., apparatus type including volume, surface tension, ionic strength, viscosity, and pH of the medium; as well as by the hydrodynamic conditions).

Dissolution profiles over a relevant pH range should be obtained under sink conditions, i.e., wherein the drug concentration in the dissolution medium is at least 3 times lower than its saturation solubility as outlined by USP <1092>. Characterization of formulations that are insoluble in aqueous systems may require the addition of other surfactants such as sodium lauryl sulfate. Measuring the dissolution rate of a drug in aqueous systems is a useful test for the comparison of different formulations or for drug product quality evaluation. To predict in vivo absorption behavior, dissolution/solubility testing should be carried out under physiological conditions. Simulated gastric fluid (SGF) or simulated intestinal fluids (SIF) are common media in such studies. A bio-relevant gastrointestinal media, simulating the fasted and fed states, may also be used (Dressman and Reppas, 2000; Kostewicz et al., 2002; Marques 2004).

Cannabinoids are generally highly lipophilic and thus have a very low water solubility. For instance, cannabidiol (CBD) is practically insoluble in aqueous media at all physiological pHs (Log P=5.8), and has an extremely low oral bioavailability (about 6%) due to its poor water solubility (0.7 μg/mL) and extensive metabolism. Considering these physical-chemical properties, solubility enhancement is required so as to deliver CBD in a bioavailable and efficient form.

### SUMMARY OF INVENTION

It has now been found, in accordance with the present invention, that a delivery system comprising D-α-tocopheryl polyethylene glycol 1000 succinate (TPGS; vitamin E TPGS; tocofersolan; α-hydro-ω-{[4-oxo-4-({(2R)-2,5,7,8-tetramethyl-2-[(4R,8R)-4,8,12-trimethyltridecyl]-3,4-dihydro-2H-1-benzopyran-6-yl}oxy)butanoyl]oxy}poly(oxyethylene) is capable of significantly improving CBD water solubility and is therefore expected to remarkably improve CBD bioavailability upon oral administration as well. As specifically shown, tablets comprising CBD and TPGS (also referred to herein as “CBD-176-66-TB”) released about three times more CBD (82%) than control tablets containing the same amount of CBD but no delivery system, in Fasted State Simulating Intestinal Fluids (FaSSIF) media, simulating gastrointestinal fluids. Moreover, tablets comprising CBD in a delivery system as referred to hereinabove, e.g., those depicted in Tables 1-9 herein, were found to be stable for at least six months following their storage at 40° C.±2° C. and 75±5% relative humidity (RH).

In one aspect, the present invention thus relates to a composition comprising a cannabinoid, e.g., CBD or an enantiomer, diastereomer, or mixture thereof, as an active agent, and TPGS, e.g., wherein the ratio between said cannabinoid and said TPGS is from about 50:1 to about 1:50, from about 10:1 to about 1:10, or from about 1:1 to about 1:5, by weight, respectively. In certain embodiments, said composition further comprises an excipient such as polyethylene glycol (PEG) and/or an antioxidant. Such a composition may be formulated as a pharmaceutical or nutraceutical composition, optionally further comprising a pharmaceutically- or nutraceutically-acceptable carrier accordingly.

In a particular such aspect, the composition of the present invention is a nutraceutical composition, further comprising at least one additional agent and optionally a binder. In certain embodiments, the composition comprises said binder, and said at least one additional active agent is a biomass of a higher Basidiomycetes mushroom or a combination thereof, or an extract obtained therefrom. Such extracts may be obtained by extracting said mushroom or combination thereof with an organic or inorganic solvent. Particular such extracts are obtained by extracting said mushroom or combination thereof with water, e.g., distilled water, at a temperature of about 60-90° C., about 70-85° C., or about 80° C.

Particular such compositions are those wherein said at least one additional agent is an extract obtained from a higher Basidiomycetes mushroom or a combination thereof, by extracting said mushroom or a combination thereof, e.g., with water as defined above, and at least one of said higher Basidiomycetes mushrooms is selected from:
(i) Coprinus comatus (O. F. Mull.) Pers. deposited under the Budapest Treaty with the Centraalbureau voor Schimmelcultures (CBS) under Accession No. CBS 146994 (hereinafter Coprinus comatus CBS 146994);
(ii) Flammulina velutipes (Curtis) Singer deposited under the Budapest Treaty with the CBS under Accession No. CBS 146995 (hereinafter Flammulina velutipes CBS 146995);
(iii) Ganoderma lucidum (Curtis: Fr.) P. Karst. deposited under the Budapest Treaty with the CBS under Accession No. CBS 146996 (hereinafter Ganoderma lucidum CBS 146996);
(iv) Grifola frondosa (Dicks.) Gray deposited under the Budapest Treaty with the CBS under Accession No. CBS 146997 (hereinafter Grifola frondosa CBS 146997);
(v) Hericium erinaceus (Bull.: Fr.) Pers. deposited under the Budapest Treaty with the CBS under Accession No. CBS 146998 (hereinafter Hericium erinaceus CBS 146998);
(vi) Pleurotus ostreatus (Jacq.: Fr.) Kummer deposited under the Budapest Treaty with the CBS under Accession No. CBS 146999 (hereinafter Pleurotus ostreatus CBS 146999); and
(vii) Trametes versicolor (L.: Fr.) Lloyd. deposited under the Budapest Treaty with the CBS under Accession No. CBS 147000 (hereinafter Trametes versicolor CBS 147000),
   optionally wherein at least another one of said higher Basidiomycetes mushrooms is selected from Agaricus brasiliensis, Auricularia aricula-judae, Cordyceps militaris, Ganoderma tsugae, Hypsizygus marmoreus, Inonotus obliquus, Lentinus edodes, Pleurotus eryngii, and Tremella fuciformis.

More particular such compositions are those wherein said combination of higher Basidiomycetes mushrooms includes:
(i) Coprinus comatus CBS 146994, Lentinus edodes, and Tremella fuciformis, preferably at about equal weights;
(ii) Flammulina velutipes CBS 146995, Ganoderma lucidum CBS 146996, Lentinus edodes, and Trametes versicolor CBS 147000, preferably at about equal weights;
(iii) Cordyceps militaris, Ganoderma lucidum CBS 146996, and Inonotus obliquus, preferably at weight ratio of about 1:1:2, respectively;
(iv) Grifola frondosa CBS 146997, Lentinus edodes, Pleurotus eryngii, and Trametes versicolor CBS 147000, preferably at about equal weights;
(v) Auricularia aricula-judae, Ganoderma lucidum CBS 146996, Lentinus edodes, Pleurotus ostreatus CBS 146999, and Trametes versicolor CBS 147000, preferably at weight ratio of about 1.5:1.5:1.5:4:1.5, respectively;
(vi) Auricularia aricula-judae, Ganoderma lucidum CBS 146996, Lentinus edodes, Pleurotus ostreatus CBS 146999, and Trametes versicolor CBS 147000, preferably at weight ratio of about 2:2:1.5:3:1.5, respectively;
(vii) Flammulina velutipes CBS 146995, Ganoderma lucidum CBS 146996, Hypsizygus marmoreus, Lentinus edodes, and Trametes versicolor CBS 147000, preferably at about equal weights;
(viii) Cordyceps militaris, Ganoderma lucidum CBS 146996, and Inonotus obliquus, preferably at weight ratio of about 3:4:3, respectively;
(ix) Cordyceps militaris, Ganoderma lucidum CBS 146996, and Hericium erinaceus CBS 146998, preferably at weight ratio of about 3:3:4, respectively;
(x) Cordyceps militaris, Ganoderma lucidum CBS 146996, and Ganoderma tsugae, preferably at weight ratio of about 2:1:1, respectively;
(xi) Ganoderma lucidum CBS 146996, and of Hericium erinaceus CBS 146998, preferably at weight ratio of about 1:3, respectively;
(xii) Agaricus brasiliensis, Coprinus comatus CBS 146994, Cordyceps militaris, Lentinus edodes, and Pleurotus ostreatus CBS 146999, preferably at weight ratio of about 1.5:1.5:1.5:4:1.5, respectively;
(xiii) Agaricus brasiliensis, Coprinus comatus CBS 146994, Ganoderma lucidum CBS 146996, and Tremella fuciformis, preferably at weight ratio of about 1:2:1:1, respectively;
(xiv) Agaricus brasiliensis, Cordyceps militaris, Ganoderma lucidum CBS 146996, Grifola frondosa CBS 146997, and Tremella fuciformis, preferably at about equal weights; or
(xv) Cordyceps militaris, Grifola frondosa CBS 146997, Tremella fuciformis, Lentinus edodes, and Ganoderma lucidum CBS 146996, preferably at about equal weights;
(xvi) Pleurotus ostreatus CBS 146999, Ganoderma lucidum CBS 146996, Auricularia aricula-judae, Grifola frondosa CBS 146997, and Lentinus edodes, preferably at weight ratio of about 4:1.5:1.5:1.5:1.5, respectively;
(xvii) Inonotus obliquus, Lentinus edodes, and Ganoderma lucidum CBS 146996, preferably at weight ratio of about 2:1:1, respectively;
(xviii) Pleurotus ostreatus CBS 146999, Ganoderma lucidum CBS 146996, Auricularia aricula-judae, Grifola frondosa CBS 146997, and Lentinus edodes, preferably at weight ratio of about 3:2:2:1.5:1.5, respectively;
(xix) Trametes versicolor CBS 147000, Cordyceps militaris, Flammulina velutipes CBS 146995, Ganoderma lucidum CBS 146996, and Lentinus edodes, preferably at about equal weights; or
(xx) Ganoderma lucidum CBS 146996 and Cordyceps militaris, preferably at about equal weights.

The composition of the present invention may be in the form of a liquid, e.g., a solution in an edible solvent, tincture, syrup, or elixir; a semi-solid; or a solid such as tablets, caplets, pills, troches, lozenges, dispersible powder or granules, hard or soft capsules, and sachets.

In another aspect, disclosed herein is a drink, beverage, or food supplement, comprising a composition as defined above.

### BRIEF DESCRIPTION OF DRAWINGS

FIG. 1 shows the dissolution profile of CBD from a CBD-containing nutraceutical composition as disclosed herein (“CBD-176-66-TB”) and from a control composition (“CBD-176-68-TB”), both formulated as tablets, in Fasted State Simulating Intestinal Fluids (FaSSIF) media.

### DETAILED DESCRIPTION OF THE INVENTION

In one aspect, the present invention relates to a composition comprising a cannabinoid as an active agent and TPGS.

The term “cannabinoid” as used herein refers to a chemical compound acting on cannabinoid receptors, i.e., a cannabinoid type 1 (CB1) and/or cannabinoid type 2 (CB2) receptor agonist. Ligands for these receptor proteins include the endocannabinoids produced naturally in the body; the phytocannabinoids found in Cannabis sativa, Cannabis indica, Cannabis ruderalis, and some other plants; and synthetic cannabinoids. The cannabinoid comprised within the composition of the invention may be derived from a Cannabis extract using any suitable extraction and purification procedures known in the art, or may alternatively be synthesized following any one of the procedures disclosed in the literature.

In certain embodiments, the cannabinoid comprised within the composition of the invention is selected from cannabidiol (CBD), cannabidiolic acid (CBDA), cannabidiphorol (CBDP), cannabidivarin (CBDV), cannabidivarinic acid (CBDVA), cannabidiol monomethyl ether (CBDM), cannabidiol-C4 (CBD-C4), cannabidiorcol (CBD-C1), Δ⁹-tetrahydrocannabinol (Δ⁹-THC), Δ⁹-tetrahydrocannabinolic acid (Δ⁹-THCA), Δ⁹-tetrahydrocannabivarin (Δ⁹-THCV), Δ⁹-THCVA, Δ⁸-THC, Δ⁸-THCA, Δ⁸-THCV, Δ⁸-THCVA, iso-tetrahydrocannabinol-type (iso-THC), cannabinol (CBN), cannabinolic acid (CBNA), cannabinol-C4 (CBN-C4), cannabinol-C2 (CBN-C2), cannabiorcol (CBN-C1), cannabinol methyl ether (CBNM), cannabinodiol (CBND), cannabigerol (CBG), cannabigerovarin (CBGV), cannabigerolic acid (CBGA), cannabigerovarinic acid (CBGVA), cannabigerol monomethyl ether (CBGM), cannabigerolic acid monomethyl ether (CBGAM), cannabichromene (CBC), cannabichromenic acid (CBCA), cannabichromevarin (CBCV), cannabichromevarinic acid (CBCVA), cannabichromanon (CBCN), cannabicyclol (CBL), cannabicyclolic acid (CBLA), cannabicyclovarin (CBLV), cannabivarin (CBV), cannabivarinic acid (CBVA), cannabielsoin (CBE), cannabielsoic acid A (CBEA-A), cannabielsoic acid B (CBEA-B), cannabitriol (CBT), cannabitriolvarin (CBTV), ethoxy-cannabitiolvarin (CBTVE), cannabifuran (CBF), dehydrocannabifuran (DCBF), cannabiripsol (CBR), an enantiomer, diastereomer, or pharmaceutically acceptable salt thereof, or a mixture thereof.

In particular embodiments, the cannabinoid comprised within the composition of the invention is CBD (2-[(1R,6R)-6-isopropenyl-3-methylcyclohex-2-en-1-yl]-5-pentylbenzene-1,3-diol), or an enantiomer, diastereomer, or a mixture, e.g., racemate, thereof, preferably CBD; or a Cannabis plant extract, e.g., Cannabis Sativa extract, comprising CBD, or said enantiomer, diastereomer, or mixture thereof. According to the literature, CBD exhibits various therapeutic effects, and is currently mostly used for seizure disorder (epilepsy), as well as for other indications including anxiety, pain, the muscle disorder called dystonia, Parkinson disease, and Crohn disease, although with no sufficient scientific evidence to support these uses.

CBD has two stereogenic centers, i.e., at positions 3 and 4 of the cyclohexenyl ring, and may accordingly exist as an enantiomer, i.e., an optical isomer (R or S, which may have an optical purity of 90%, 95%, 99% or more), racemate, i.e., an optically inactive mixture having equal amounts of R and S enantiomers, a diastereoisomer, or a mixture thereof. The present invention encompasses compositions wherein the active agent is any one of such enantiomers, isomers and mixtures thereof.

CBD may be synthesized following any one of the procedures known in the art, e.g., by acid condensation of p-mentha-2,8-dien-1-ol with olivetol. Optically active forms of CBD may be prepared using any one of the methods disclosed in the art, e.g., by resolution of the racemic form by recrystallization techniques; chiral synthesis; extraction with chiral solvents; or chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

In particular embodiments, the composition of the invention comprises a Cannabis plant extract comprising a cannabinoid as referred to hereinabove, e.g., CBD. Such an extract may be obtained utilizing any method or technique known in the art, or may alternatively be a commercially available product. In specific such embodiments, the Cannabis plant extract is obtainable from the seeds of said Cannabis plant such as hemp oil (hempseed oil).

TPGS is a water-soluble derivative of vitamin E having amphiphilic properties, with a polar hydrophilic head (polyethylene glycol 1000; PEG₁₀₀₀) and a lipophilic tail (phytyl chain of D-α-tocopherol), a hydrophilic/lipophilic balance of 13, and a critical micelle concentration (CMC) of 0.02 weight % at 37° C. It is therefore used as a multirole excipient for pharmaceutical and nutrients delivery innovation. In particular, it is a non-ionic surfactant used as a solubilizer of poorly soluble drugs, absorption enhancer, emulsifier, stabilizer of amorphous solid dispersion (ASD), vehicle for lipid-based drug formulation, and/or antioxidant.

TPGS has shown to improve bioavailability of poorly absorbed drugs, vitamins, and micro-nutrients by acting as an absorption and permeability enhancer, and to develop self-emulsifying drug delivery system (SEEDS) for poorly soluble drugs by acting as an emulsifier.

TPGS is safe for use in animals and humans based on reported toxicological studies (studies in humans included dosing of cholestatic children at 25 IU/kg/day, which is equivalent to 64 mg TPGS/kg/day, for over two years), has a self-affirmed GRAS (generally recognized as safe) status when used as an oral dietary supplement of vitamin E, and is not genotoxic. The United States Pharmacopeia (USP) published a monograph for TPGS in the USP/NF Supplement #9 dated of Nov. 15, 1998. Studies to assess the safety and bioavailability of TPGS for use in food, particularly for nutritional/medical purposes, have been conducted by the European Food Safety Authority (EFSA) (EFSA Journal, 2007, 490, 1-20). From toxicology studies, an overall no-observed-adverse-effect level (NOAEL) of 1000 mg/kg body weight per day can be derived.

The TPGS comprised within the composition of the invention act, in fact, as a delivery system aimed at enhancing/improving the water solubility and consequently bioavailability of the active agent, i.e., the cannabinoid. Specifically, the TPGS forms various micellar liquid crystalline forms with water, and the cannabinoid, e.g., CBD, is solubilized by such micellar liquid crystals and/or by a self-emulsification mechanism in the presence of water and TPGS.

In certain embodiments, the weight ratio between the cannabinoid and the TPGS comprised within the composition of the invention, as defined in any one of the embodiments above, is from about 50:1 to about 1:50, e.g., from about 30:1 to about 1:30, from about 20:1 to about 1:20, from about 10:1 to about 1:10, from about 5:1 to about 1:5, or about 1:1, about 1:1.5, about 1:2, about 1:2.5, about 1:3, about 1:3.5, about 1:4, or about 1.4.5, by weight, respectively.

In certain embodiments, the composition of the invention according to any one of the embodiments above further comprises at least one excipient such as polyethylene glycol (PEG) and polyvinylpyrrolidone (povidone, PVP). The addition of PEG, which has a low toxicity with systemic absorption of less than 0.5%, as a free ingredient, i.e., in addition to the PEG₁₀₀₀ being the polar hydrophilic head of the TPGS, improves TPGS ability to solubilize the cannabinoid in a mixed micellular system and makes it bioavailable. In particular embodiments, the molecular weight of said PEG is up to about 50 kDa, e.g., from about 2 kDa to about 5 kDa, 10 kDa, 20 kDa, 30 kDa, 40 kDa, or 50 kDa, but preferably about 2 kDa, 3 kDa, 4 kDa, or 5 kDa. In particular embodiments, the ratio between the cannabinoid, e.g., CBD, and the excipient comprised within the composition of the invention is from about 1:0.1 to about 1:1, e.g., from about 1:0.2 to about 1:1, from about 1:0.25 to about 1:0.95, from about 1:0.3 to about 1:0.9, from about 1:0.35 to about 1:0.85, or from about 1:0.4 to about 1:0.8, by weight, respectively.

In certain embodiments, the composition of the invention according to any one of the embodiments above further comprises at least one antioxidant, i.e., a compound capable of preventing, delaying, or inhibiting oxidative degradation of an oxidizable compound, and thus inhibiting or limiting the formation of free radicals, which lead to a chain reaction that may eventually damage said oxidizable compound.

Examples of antioxidants include, without being limited to, butylated hydroxytoluene (BHT); an o-quinone scavenger such as ascorbic acid (vitamin C) or a salt thereof (e.g., Na-ascorbate); ascorbic acid derivative such as a stereoisomer thereof (e.g., erythorbic acid) and a fatty ester thereof (e.g., ascorbic acid-6-palmitate); methylated phenols such as tocopherol (e.g., α-tocopherol, β-tocopherol, γ-tocopherol, and δ-tocopherol) or a derivative thereof such as a tocotrienol; 4-hexylresorcinol; propyl gallate; synthetic antioxidants such as butylated hydroxyanisole (BHA); tyrosinase inhibitors such as captopril; and sulfur containing o-quinone scavengers, e.g., L-methionine (also referred to herein as methionine) (see, e.g., Bittner 2006), N-acetylnethionine, L-cysteine (also referred to herein as cysteine), N-acetyl cysteine (NAC), and gluthatione (GSH); Cu²⁺ chelators such as Na₂-EDTA, Na₂-EDTA-Ca, DMSA (succimer), D-penicillamine (DPA), trientine-HCl, dimercaprol, clioquinol, sodium thiosulfate, triethylenetetramine (TETA), tetraethylenepentamine (TEPA), curcumin, neocuproine, tannin, and/or cuprizone; sulfite salts such as sodium hydrogen sulfite or sodium metabisulfite; and rosemary extract.

In particular embodiments wherein one or more antioxidants are included in the composition of the invention, the ratio between the cannabinoid, e.g., CBD, and the antioxidant comprised within the composition is from about 1:0.1 to about 1:10, e.g., from about 1:0.3 to about 1:7, from about 1:0.5 to about 1:5, from about 1:0.8 to about 1:2, or about 1:1, by weight, respectively.

In certain embodiments, the cannabinoid comprised within the composition of the invention is CBD, and the ratio between said CBD and said TPGS in said composition is from about 10:1 to about 1:10, from about 5:1 to about 1:5, or about 1:1, about 1:1.5, about 1:2, about 1:2.5, about 1:3, about 1:3.5, about 1:4, or about 1.4.5, by weight, respectively. Particular such compositions further comprise PEG as an excipient, and BHT as an antioxidant, wherein the ratio between said CBD and said PEG is from about 1:0.1 to about 1:1, e.g., from about 1:0.2 to about 1:1, from about 1:0.25 to about 1:0.95, from about 1:0.3 to about 1:0.9, from about 1:0.35 to about 1:0.85, or from about 1:0.4 to about 1:0.8, by weight, respectively; and the ratio between said CBD and said BHT is from about 1:0.1 to about 1:10, e.g., from about 1:0.3 to about 1:7, from about 1:0.5 to about 1:5, from about 1:0.8 to about 1:2, or about 1:1, by weight, respectively.

The compositions disclosed herein, according to any one of the embodiments above, may be formulated as pharmaceutical- or nutraceutical compositions optionally further comprising one or more suitable carriers and/or excipients.

In certain embodiments, the composition disclosed is a pharmaceutical composition optionally further comprising a pharmaceutically acceptable carrier and/or excipient.

The term “pharmaceutically acceptable carrier” or “pharmaceutically acceptable excipient” as used herein interchangeably refers to any non-active ingredient such as a solvent, dispersion medium, preservative, antioxidant, coating, isotonic and absorption delaying agent, and the like, that is compatible with pharmaceutical administration, and does not produce an adverse, allergic, or other untoward reaction when administered to a mammal or human as appropriate.

For human administration, compositions should meet sterility, pyrogenicity, and general safety and purity standards as required by, e.g., the U.S. Food and Drug Administration (FDA), the European Medicines Agency (EMA), the Therapeutic Goods Administration (Australia), the Medicines and Healthcare products Regulatory Agency (United Kingdom), or the Pharmaceuticals and Medical Devices Agency (Japan).

Pharmaceutical compositions as disclosed herein may be prepared by conventional techniques, e.g., as described in Remington: The Science and Practice of Pharmacy, 19^{th} Ed., 1995. The compositions can be prepared, e.g., by uniformly and intimately bringing the active agents into association with a liquid carrier, a finely divided solid carrier, or both, and then, if necessary, shaping the product into the desired formulation.

The pharmaceutical composition of the invention may be formulated for any suitable administration route, but they are preferably formulated for oral, sublingual, buccal, or sublabial administration. The compositions may be in the form of a liquid, e.g., a solution in an edible solvent such as ethanol, tincture, syrup, or elixir; a semi-solid; or a solid such as tablets, caplets, pills, troches, lozenges, dispersible powder or granules, hard or soft capsules, and sachets. In certain embodiments, the compositions are in the form of matrix tablets, wherein the release of a soluble cannabinoid is controlled by having the cannabinoid diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other embodiments, the tablets are formulated as bi- or multi-layer tablets, made up of two or more distinct layers of granulation compressed together with the individual layers lying one on top of another, with each separate layer containing a different active agent. Bilayer tablets have the appearance of a sandwich since the edge of each layer or zone is exposed.

In certain embodiments, the invention provides a pharmaceutical composition for oral administration, which is solid and may be in the form of granulate, granules, grains, beads or pellets, mixed and filled into capsules or sachets, or compressed to tablets by conventional methods. In some particular embodiments, the pharmaceutical composition is in the form of a bi- or multilayer tablet, in which each one of the layers comprise the active agent, and the layers are optionally separated by an intermediate, inactive layer, e.g., a layer comprising one or more disintegrants.

Another contemplated formulation is depot systems, based on biodegradable polymers. As the polymer degrades, the active agent(s) is slowly released. The most common class of biodegradable polymers is the hydrolytically labile polyesters prepared from lactic acid, glycolic acid, or combinations of these two molecules. Polymers prepared from these individual monomers include poly (D,L-lactide) (PLA), poly (glycolide) (PGA), and the copolymer poly (D,L-lactide-co-glycolide) (PLG).

Pharmaceutical compositions for oral administration may be prepared according to any method known to the art and may further comprise one or more agents selected from sweetening agents, flavoring agents, coloring agents and preserving agents in order to provide pharmaceutically elegant and palatable preparations. Tablets contain the active agents in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of oil-in-water emulsion.

Useful dosage forms of the pharmaceutical compositions include orally disintegrating systems including, but not limited to, solid, semi-solid and liquid systems including disintegrating or dissolving tablets, soft or hard capsules, gels, fast dispersing dosage forms, controlled dispersing dosage forms, caplets, films, wafers, ovules, granules, buccal/mucoadhesive patches, powders, freeze dried (lyophilized) wafers, chewable tablets which disintegrate with saliva in the buccal/mouth cavity and combinations thereof. Useful films include, but are not limited to, single layer stand-alone films and dry multiple layer stand-alone films.

Pharmaceutical compositions for oral administration may be formulated for controlled release of the active agent, i.e., the cannabinoid. Such compositions may be formulated as controlled-release matrix, e.g., as controlled-release matrix tablets in which the release of a soluble active agent is controlled by having the active diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other configurations, the compositions comprise the active agent formulated for controlled release in microencapsulated dosage form, in which small droplets of the active agent are surrounded by a coating or a membrane to form particles in the range of a few micrometers to a few millimeters.

In particular embodiments, pharmaceutical compositions for oral administration may be formulated so as to inhibit the release of the active agent in the stomach, i.e., delay the release of said active agent until at least a portion of the dosage form has traversed the stomach, in order to avoid the acidity of the gastric contents from hydrolyzing the active agent. Particular such compositions are those wherein the active agent is coated by a pH-dependent enteric-coating polymer. Examples of pH-dependent enteric-coating polymer include, without being limited to, Eudragit® S (poly(methacrylicacid, methylmethacrylate), 1:2), Eudragit® L 55 (poly (methacrylicacid, ethylacrylate), 1:1), Kollicoat® (poly(methacrylicacid, ethylacrylate), 1:1), hydroxypropyl methylcellulose phthalate (HPMCP), alginates, carboxymethylcellulose, and combinations thereof. The pH-dependent enteric-coating polymer may be present in the composition in an amount from about 10% to about 95% by weight of the entire composition.

The pharmaceutical composition of the invention may comprise one or more pharmaceutically acceptable excipients. For example, a tablet may comprise at least one filler, e.g., lactose, ethylcellulose, microcrystalline cellulose, silicified microcrystalline cellulose; at least one disintegrant, e.g., cross-linked polyvinylpyrrolidinone; at least one binder, e.g., polyvinylpyridone, hydroxypropylmethyl cellulose; at least one surfactant, e.g., sodium laurylsulfate; at least one glidant, e.g., colloidal silicon dioxide; and at least one lubricant, e.g., magnesium stearate.

The pharmaceutical composition of the invention may be in the form of a sterile injectable aqueous or oleaginous suspension, which may be formulated according to the known art using suitable dispersing, wetting or suspending agents. The sterile injectable preparation may also be an injectable solution or suspension in a non-toxic parenterally acceptable diluent or solvent. Acceptable vehicles and solvents that may be employed include, without limiting, water, Ringer's solution, polyethylene glycol (PEG), 2-hydroxypropyl-β-cyclodextrin (HPCD), a surfactant such as Tween-80, and isotonic sodium chloride solution.

In certain embodiments, the composition disclosed is a nutraceutical composition optionally further comprising a nutraceutically acceptable carrier and/or excipient, formulated, e.g., for oral, sublingual, buccal, or sublabial administration.

In certain embodiments, the nutraceutical composition of the invention further comprises at least one, i.e., one, two, three, or more, additional active agents, and optionally at least one binder.

The term “binder” or “binding agent” used herein interchangeably refers to any material or substance that holds or draws other materials together to form a mechanically and/or chemically cohesive whole, by adhesion or cohesion. More specifically, binders are liquid or dough-like substances that harden by a chemical or physical process and bind fibers, filler powder and other particles added into it. Examples of binding agents include, without being limited to, a saccharide such as sucrose and glucose (e.g., liquid glucose); a polysaccharide such as starch (e.g., starch paste and pregelatinized starch) and cellulose; a natural binder such as acacia (also known as gum Arabic), tragacanth, gelatin, and alginic acid; or a synthetic or semi-synthetic polymer such as a cellulose-based polymer (e.g., methylcellulose, ethylcellulose, hydroxypropyl cellulose, hydroxypropyl methylcellulose (HPMC), and sodium carboxymethyl cellulose), polyvinyl pyrrolidone (PVP), polyvinyl alcohol, PEG, and a polymethacrylate (e.g., poly(methyl methacrylate) (PMMA)).

Based on estimations, mushrooms constitute at least 12,000 species worldwide, wherein about 2,000 of them are reported as edible. About 35 edible mushroom species are commercially cultivated, and nearly 200 wild species are being used for medicinal purposes (Üstün et al., 2018; Beulah et al., 2013). Many researchers have documented that edible mushrooms are the source of a variety of nutraceutical compounds such as polysaccharides (β-glucans), dietary fibers, unsaturated fatty acids, terpenes, peptides, glycoproteins, alcohols, mineral elements and antioxidants like phenolic compounds, tocopherols, and ascorbic acid (Pardeshi and Pardeshi, 2009).

For instance, β-glucans (beta-glucans) comprise a group of β-D-glucose polysaccharides naturally occurring in the cell walls of cereals, bacteria, and fungi, with significantly different physicochemical properties depending on their source. Typically, β-glucans form a linear backbone with 1-3 β-glycosidic bonds but vary with respect to molecular mass, solubility, viscosity, branching structure, and gelation properties, causing diverse physiological effects in animals. At dietary intake levels of at least 3 g/day, oat fiber β-glucan decreases blood levels of low-density lipoprotein (LDL) cholesterol and so may reduce the risk of cardiovascular diseases.

Mushrooms are excellent functional foods containing, e.g., ergothioneine, vitamin D2, vitamin B1, selenium, and iron, wherein the amount and bioavailability of each one of the nutrients and bioactive biomolecules primarily depend on the mushroom variety (Ruiz-Rodriguez et al., 2009; Yokota et al., 2016). Higher Basidiomycetes mushrooms contain biologically active compounds in fruit bodies, cultured mycelium, and cultured broth (Wasser 2014). Thus, they might be used directly in diet and promote health, taking advantage of the additive and synergistic effects of all the bioactive compounds they contain (Vaz et al., 2010; Pereira et al., 2012, Badalyan and Singh 2014). The presence of specific bioactive compounds makes these mushrooms valuable, from strengthening of the immune system to the treatment and prevention of life-threatening diseases such as heart disease, hypertension, cerebral stroke, and cancer. Mushrooms are also known to exhibit antiviral, antibacterial, antifungal, anti-inflammatory, anti-tumor and immunomodulating, cardiovascular, hepatoprotective, antidiabetic, hypolipidemic, antithrombotic, and hypotensive effects (Wasser and Weis 1999a; Wasser and Weis 1999b; Singh 2017). The properties and mechanisms of extracts and bioactive compounds from mushrooms have already been evaluated both in vitro and in vivo, including in human (Rathee et al., 2012; Roupas et al., 2012; Giavasis 2014a; Giavasis 2014b; Kothari et al., 2018).

In certain embodiments, the nutraceutical composition of the invention comprises at least one additional active agent and a binder, wherein said at least one additional active agent is a biomass of a higher Basidiomycetes mushroom or a combination thereof, or an extract obtained by extracting said mushroom or combination thereof with an organic or inorganic solvent (also referred to herein as “Cannabinoid-mushroom-based nutraceutical composition”). Particular such extracts are obtained by extracting said higher Basidiomycetes mushroom or combination thereof with water, e.g., distilled water, at a temperature of about 60-90° C., 70-85° C., or 80° C., and are rich in amino acids including the twenty-two amino acids naturally occurring in proteins, i.e., aspartic acid, tyrosine, leucine, tryptophan, arginine, valine, glutamic acid, methionine, phenylalanine, serine, alanine, glutamine, glycine, proline, threonine, asparagine, lysine, histidine, isoleucine, cysteine, selenocysteine, and pyrrolysine, as well as 7-aminobutyric acid (GABA) and ergothioneine.

In particular such embodiments, said at least one additional agent is an extract obtained from a higher Basidiomycetes mushroom or a combination thereof, by extracting said mushroom or a combination thereof with water as defined above, and at least one of said higher Basidiomycetes mushrooms is selected from Coprinus comatus CBS 146994; Flammulina velutipes CBS 146995; Ganoderma lucidum CBS 146996; Grifola frondosa CBS 146997; Hericium erinaceus CBS 146998; Pleurotus ostreatus CBS 146999; and Trametes versicolor CBS 147000. In more particular such embodiments, at least another one of said higher Basidiomycetes mushrooms is selected from Agaricus brasiliensis, Auricularia aricula-judae, Cordyceps militaris, Ganoderma tsugae, Hypsizygus marmoreus, Inonotus obliquus, Lentinus edodes, Pleurotus eryngii, and Tremella fuciformis. Such extracts are the subject-matter of International Patent Application No. PCT/IL2022/050284, filed Mar. 14, 2022, herein incorporated by reference in its entirety as if fully disclosed herein.

Examples of such cannabinoid-mushroom-based nutraceutical compositions, without being limited to, are those wherein said combination of higher Basidiomycetes mushrooms include:
(i) Coprinus comatus CBS 146994, Lentinus edodes, and Tremella fuciformis, preferably at about equal weights;
(ii) Flammulina velutipes CBS 146995, Ganoderma lucidum CBS 146996, Lentinus edodes, and Trametes versicolor CBS 147000, preferably at about equal weights;
(iii) Cordyceps militaris, Ganoderma lucidum CBS 146996, and Inonotus obliquus, preferably at weight ratio of about 1:1:2, respectively;
(iv) Grifola frondosa CBS 146997, Lentinus edodes, Pleurotus eryngii, and Trametes versicolor CBS 147000, preferably at about equal weights;
(v) Auricularia aricula-judae, Ganoderma lucidum CBS 146996, Lentinus edodes, Pleurotus ostreatus CBS 146999, and Trametes versicolor CBS 147000, preferably at weight ratio of about 1.5:1.5:1.5:4:1.5, respectively;
(vi) Auricularia aricula-judae, Ganoderma lucidum CBS 146996, Lentinus edodes, Pleurotus ostreatus CBS 146999, and Trametes versicolor CBS 147000, preferably at weight ratio of about 2:2:1.5:3:1.5, respectively;
(vii) Flammulina velutipes CBS 146995, Ganoderma lucidum CBS 146996, Hypsizygus marmoreus, Lentinus edodes, and Trametes versicolor CBS 147000, preferably at about equal weights;
(viii) Cordyceps militaris, Ganoderma lucidum CBS 146996, and Inonotus obliquus, preferably at weight ratio of about 3:4:3, respectively;
(ix) Cordyceps militaris, Ganoderma lucidum CBS 146996, and Hericium erinaceus CBS 146998, preferably at weight ratio of about 3:3:4, respectively;
(x) Cordyceps militaris, Ganoderma lucidum CBS 146996, and Ganoderma tsugae, preferably at weight ratio of about 2:1:1, respectively;
(xi) Ganoderma lucidum CBS 146996, and of Hericium erinaceus CBS 146998, preferably at weight ratio of about 1:3, respectively;
(xii) Agaricus brasiliensis, Coprinus comatus CBS 146994, Cordyceps militaris, Lentinus edodes, and Pleurotus ostreatus CBS 146999, preferably at weight ratio of about 1.5:1.5:1.5:4:1.5, respectively;
(xiii) Agaricus brasiliensis, Coprinus comatus CBS 146994, Ganoderma lucidum CBS 146996, and Tremella fuciformis, preferably at weight ratio of about 1:2:1:1, respectively;
(xiv) Agaricus brasiliensis, Cordyceps militaris, Ganoderma lucidum CBS 146996, Grifola frondosa CBS 146997, and Tremella fuciformis, preferably at about equal weights;
(xv) Cordyceps militaris, Grifola frondosa CBS 146997, Tremella fuciformis, Lentinus edodes, and Ganoderma lucidum CBS 146996, preferably at about equal weights;
(xvi) Pleurotus ostreatus CBS 146999, Ganoderma lucidum CBS 146996, Auricularia aricula-judae, Grifola frondosa CBS 146997, and Lentinus edodes, preferably at weight ratio of about 4:1.5:1.5:1.5:1.5, respectively; (xvii) Inonotus obliquus, Lentinus edodes, and Ganoderma lucidum CBS 146996, preferably at weight ratio of about 2:1:1, respectively;
(xviii) Pleurotus ostreatus CBS 146999, Ganoderma lucidum CBS 146996, Auricularia aricula-judae, Grifola frondosa CBS 146997, and Lentinus edodes, preferably at weight ratio of about 3:2:2:1.5:1.5, respectively;
(xix) Trametes versicolor CBS 147000, Cordyceps militaris, Flammulina velutipes CBS 146995, Ganoderma lucidum CBS 146996, and Lentinus edodes, preferably at about equal weights; or
(xx) Ganoderma lucidum CBS 146996 and Cordyceps militaris, preferably at about equal weights.

In certain embodiments, disclosed herein is a cannabinoid-mushroom-based nutraceutical composition according to any one of the embodiments above, wherein said cannabinoid constitutes from about 0.5% to about 3% by weight of said composition; said binder constitutes from about 1.5% to about 4% by weight of said composition; and said at least one additional agent constitutes from about 50% to about 80% by weight of said composition. In particular embodiments, said composition further comprises (i) an excipient selected from PEG having a molecular weight of, e.g., about 2 kDa to about 50 kDa, preferably from about 2 kDa to about 5 kDa; and/or (ii) an antioxidant such as BHT; ascorbic acid, a stereoisomer thereof such as erythorbic acid, a fatty ester thereof, or a salt thereof; a tocopherol such as α-tocopherol, β-tocopherol, γ-tocopherol, and 6-tocopherol, or a derivative thereof such as a tocotrienol; 4-hexylresorcinol; propyl gallate; BHA; rosemary extract; and a mixture thereof. More particular such compositions are those wherein said excipient constitutes from about 0.3% to about 1.5% by weight of said composition; and/or said antioxidant constitutes from about 0.5% to about 3% by weight of said composition. In certain specific such compositions, said cannabinoid is CBD or an enantiomer, diastereomer, or mixture thereof, preferably CBD; or a Cannabis plant extract, e.g., Cannabis Sativa extract, comprising CBD or said enantiomer, diastereomer, or mixture thereof (also referred to herein as “CBD-mushroom-based nutraceutical composition”).

In certain embodiments, disclosed herein is a CBD-mushroom-based nutraceutical composition according to any one of the embodiments above, wherein said cannabinoid is CBD or an enantiomer, diastereomer, or mixture thereof; and said at least one additional agent is an extract obtained from a combination of the higher Basidiomycetes mushrooms: (i) Coprinus comatus CBS 146994, Lentinus edodes, and Tremella fuciformis, preferably at about equal weights; (ii) Flammulina velutipes CBS 146995, Ganoderma lucidum CBS 146996, Lentinus edodes, and Trametes versicolor CBS 147000, preferably at about equal weights; (iii) Cordyceps militaris, Ganoderma lucidum CBS 146996, and Inonotus obliquus, preferably at weight ratio of about 1:1:2, respectively; (iv) Grifola frondosa CBS 146997, Lentinus edodes, Pleurotus eryngii, and Trametes versicolor CBS 147000, preferably at about equal weights; (v) Auricularia aricula-judae, Ganoderma lucidum CBS 146996, Lentinus edodes, Pleurotus ostreatus CBS 146999, and Trametes versicolor CBS 147000, preferably at weight ratio of about 1.5:1.5:1.5:4:1.5, respectively; (vi) Auricularia aricula-judae, Ganoderma lucidum CBS 146996, Lentinus edodes, Pleurotus ostreatus CBS 146999, and Trametes versicolor CBS 147000, preferably at weight ratio of about 2:2:1.5:3:1.5, respectively; (vii) Flammulina velutipes CBS 146995, Ganoderma lucidum CBS 146996, Hypsizygus marmoreus, Lentinus edodes, and Trametes versicolor CBS 147000, preferably at about equal weights; (viii) Cordyceps militaris, Ganoderma lucidum CBS 146996, and Inonotus obliquus, preferably at weight ratio of about 3:4:3, respectively; (ix) Cordyceps militaris, Ganoderma lucidum CBS 146996, and Hericium erinaceus CBS 146998, preferably at weight ratio of about 3:3:4, respectively; (x) Cordyceps militaris, Ganoderma lucidum CBS 146996, and Ganoderma tsugae, preferably at weight ratio of about 2:1:1, respectively; (xi) Ganoderma lucidum CBS 146996, and of Hericium erinaceus CBS 146998, preferably at weight ratio of about 1:3, respectively; (xii) Agaricus brasiliensis, Coprinus comatus CBS 146994, Cordyceps militaris, Lentinus edodes, and Pleurotus ostreatus CBS 146999, preferably at weight ratio of about 1.5:1.5:1.5:4:1.5, respectively; (xiii) Agaricus brasiliensis, Coprinus comatus CBS 146994, Ganoderma lucidum CBS 146996, and Tremella fuciformis, preferably at weight ratio of about 1:2:1:1, respectively; (xiv) Agaricus brasiliensis, Cordyceps militaris, Ganoderma lucidum CBS 146996, Grifola frondosa CBS 146997, and Tremella fuciformis, preferably at about equal weights; (xv)Cordyceps militaris, Grifola frondosa CBS 146997, Tremella fuciformis, Lentinus edodes, and Ganoderma lucidum CBS 146996, preferably at about equal weights; (xvi) Pleurotus ostreatus CBS 146999, Ganoderma lucidum CBS 146996, Auricularia aricula-judae, Grifola frondosa CBS 146997, and Lentinus edodes, preferably at weight ratio of about 4:1.5:1.5:1.5:1.5, respectively; (xvii) Inonotus obliquus, Lentinus edodes, and Ganoderma lucidum CBS 146996, preferably at weight ratio of about 2:1:1, respectively; (xviii) Pleurotus ostreatus CBS 146999, Ganoderma lucidum CBS 146996, Auricularia aricula-judae, Grifola frondosa CBS 146997, and Lentinus edodes, preferably at weight ratio of about 3:2:2:1.5:1.5, respectively; (xix) Trametes versicolor CBS 147000, Cordyceps militaris, Flammulina velutipes CBS 146995, Ganoderma lucidum CBS 146996, and Lentinus edodes, preferably at about equal weights; or (xx) Ganoderma lucidum CBS 146996 and Cordyceps militaris, preferably at about equal weights, by extracting said combination of mushrooms with water as defined above. In particular such embodiments, said composition further comprises PEG as an excipient, and an antioxidant selected from BHT, ascorbic acid, and a mixture thereof, wherein the molecular weight of said PEG is preferably from about 2 kDa to about 50 kDa, more preferably from about 2 kDa to about 5 kDa; the amount of said PEG in said composition is from about 0.5% to about 1.5% by weight; and the amount of said antioxidant in said composition is from about 0.7% to about 1.5% by weight.

In certain such specific embodiments, disclosed herein is a CBD-mushroom-based nutraceutical composition as defined hereinabove, comprising:
(i) CBD in an amount of about 1.6% by weight; TPGS in an amount of about 1.9% by weight; PEG having a molecular weight of about 3 kDa, in an amount of about 0.8% by weight; a mixture of BHT and ascorbic acid each independently in an amount of about 0.9% by weight; and an extract obtained from a combination of the higher Basidiomycetes mushrooms Coprinus comatus CBS 146994, Lentinus edodes, and Tremella fuciformis, preferably at about equal weights (also referred to herein as “anti-aging composition”), in an amount of about 69% by weight;
(ii) CBD in an amount of about 1.6% by weight; TPGS in an amount of about 1.9% by weight; PEG having a molecular weight of about 3 kDa, in an amount of about 0.8% by weight; a mixture of BHT and ascorbic acid each independently in an amount of about 0.9% by weight; and an extract obtained from a combination of the higher Basidiomycetes mushrooms Flammulina velutipes CBS 146995, Ganoderma lucidum CBS 146996, Lentinus edodes, and Trametes versicolor CBS 147000, preferably at about equal weights (also referred to herein as “anti-oxidant composition”), in an amount of about 69% by weight;
(iii) CBD in an amount of about 1.6% by weight; vitamin E TPGS in an amount of about 1.9% by weight; PEG having a molecular weight of about 3 kDa, in an amount of about 0.8% by weight; a mixture of BHT and ascorbic acid each independently in an amount of about 0.9% by weight; and an extract obtained from a combination of the higher Basidiomycetes mushrooms Inonotus obliquus, Lentinus edodes, and Ganoderma lucidum CBS 146996, preferably at weight ratio of about 2:1:1, respectively (also referred to herein as “anti-psoriasis composition”), in an amount of about 69% by weight;
(iv) CBD in an amount of about 1.6% by weight; TPGS in an amount of about 1.9% by weight; PEG having a molecular weight of about 3 kDa, in an amount of about 0.8% by weight; BHT in an amount of about 0.9% by weight; and an extract obtained from a combination of the higher Basidiomycetes mushrooms Cordyceps militaris, Ganoderma lucidum CBS 146996, and Inonotus obliquus, preferably at weight ratio of about 1:1:2, respectively (also referred to herein as “pain killer composition”), in an amount of about 65% by weight;
(v) CBD in an amount of about 1.6% by weight; TPGS in an amount of about 1.9% by weight; PEG having a molecular weight of about 3 kDa, in an amount of about 0.8% by weight; BHT in an amount of about 0.9% by weight; and an extract obtained from a combination of the higher Basidiomycetes mushrooms Agaricus brasiliensis, Coprinus comatus CBS 146994, Cordyceps militaris, Lentinus edodes, and Pleurotus ostreatus CBS 146999, preferably at weight ratio of about 1.5:1.5:1.5:4:1.5, respectively (also referred to herein as “anti-fatty liver composition”), in an amount of about 70% by weight;
(vi) CBD in an amount of about 1.6% by weight; TPGS in an amount of about 1.9% by weight; PEG having a molecular weight of about 3 kDa, in an amount of about 0.8% by weight; BHT in an amount of about 0.9% by weight; and an extract obtained from a combination of the higher Basidiomycetes mushrooms Agaricus brasiliensis, Coprinus comatus CBS 146994, Ganoderma lucidum CBS 146996, and Tremella fuciformis, preferably at weight ratio of about 1:2:1:1, respectively (also referred to herein as “anti-diabetes composition”), in an amount of about 70% by weight;
(vii) CBD in an amount of about 1.6% by weight; TPGS in an amount of about 1.9% by weight; PEG having a molecular weight of about 3 kDa, in an amount of about 0.8% by weight; a mixture of BHT and ascorbic acid each independently in an amount of about 0.9% by weight; and an extract obtained from a combination of the higher Basidiomycetes mushrooms Cordyceps militaris, Grifola frondosa CBS 146997, Tremella fuciformis, Lentinus edodes, and Ganoderma lucidum CBS 146996, preferably at about equal weights (also referred to herein as “bone strengthener composition”), in an amount of about 60% by weight;
(viii) CBD in an amount of about 1.6% by weight; TPGS in an amount of about 2% by weight; PEG having a molecular weight of about 3 kDa, in an amount of about 0.8% by weight; BHT in an amount of about 0.9% by weight; and an extract obtained from a combination of the higher Basidiomycetes mushrooms Pleurotus ostreatus CBS 146999, Ganoderma lucidum CBS 146996, Auricularia aricula-judae, Grifola frondosa CBS 146997, and Lentinus edodes, preferably at weight ratio of about 4:1.5:1.5:1.5:1.5, respectively (also referred to herein as “anti-arrhytmic composition”), in an amount of about 70% by weight; or
(ix) CBD in an amount of about 1.2% by weight; TPGS in an amount of about 1.8% by weight; PEG having a molecular weight of about 3 kDa, in an amount of about 0.8% by weight; BHT in an amount of about 0.9% by weight; and an extract obtained from a combination of the higher Basidiomycetes mushrooms Trametes versicolor CBS 147000, Cordyceps militaris, Flammulina velutipes CBS 146995, Ganoderma lucidum CBS 146996, and Lentinus edodes, preferably at about equal weights (also referred to herein as “immunoguard composition”), in an amount of about 66% by weight.

The compositions of the invention (both the pharmaceutical and nutraceutical compositions), according to any one of the embodiments above, may be in the form of a liquid, e.g., a solution in an edible solvent such as ethanol, tincture, syrup, or elixir; a semi-solid; or a solid such as tablets, caplets, pills, troches, lozenges, dispersible powder or granules, hard or soft capsules, and sachets.

In another aspect, the present invention provides a drink, beverage, or food supplement, comprising a nutraceutical composition according to any one of the embodiments above, e.g., a cannabinoid-mushroom-based nutraceutical composition or a CBD-mushroom-based nutraceutical composition as defined in any one of the embodiments herein.

Unless otherwise indicated, all numbers expressing, e.g., amounts of components and temperature, used in this specification, are to be understood as being modified in all instances by the term “about”. Accordingly, unless indicated to the contrary, the numerical parameters set forth in this specification are approximations that may vary by up to plus or minus 10% depending upon the desired properties to be obtained by the present invention.

The invention will now be illustrated by the following non-limiting Examples.

### EXAMPLES

### Example 1. Preparation of CBD-Mushroom-Based Nutraceutical Composition in the Form of Tablets (Caplets)

Methods. Physical modification of CBD was performed by wet granulation process. CBD, vitamin E-TPGS and polyvinyl pyrrolidone, acting as both a binder and a solubilizer, were dissolved in ethanol and then mixed with a mushroom extract powder. Upon evaporation of the ethanol, dry granules, each containing the CBD and the mushrooms extract in intimate contact such that they may be dissolved and absorbed together, were formed.

Preparation of the caplets was performed at room temperature, according to the following steps:
(i) ingredients weighing and mixing;
(ii) wet-granulation with ethanol, as described above;
(iii) granulate final drying by mixing with a chopper;
(iv) granulate grinding and sieving;
(v) final blending with ex-granulate excipients;
(vi) compression of final blend to tablets;
(vii) coating with green colored cellulose-based coating system; and (viii) tablet testing.

Using the preparation protocol described above, several types of tablets, each containing a different mushroom extract powder, were prepared. Based on the specific mushroom extract powder contained, these tablets are identified herein as “anti-aging composition”, “anti-oxidant composition”, “anti-psoriasis composition”, “pain killer composition”, “anti-fatty liver composition”, “anti-diabetic composition”, “bone strengthener composition”, “anti-arrhythmic composition”, and “immunoguard composition”, and the content of each one of these compositions is summarized in Tables 1-9 (INS means International Numbering System for Food Additives).

TABLE 1
An anti-aging composition (tablet)

| | Amount | Percentage | | |
|---|---|---|---|---|
| Ingredient | (mg) | (%) | Function | INS |
| Mushroom mix: Coprinus | 900 | 69.23 | filler and functional | |
| comatus CBS 146994 (33%), | | | excipient/ingredient | |
| Lentinus edodes (33%), and | | | | |
| Tremella fuciformis (33%) | | | | |
| polyvinylpyrrolidone K30 | 30 | 2.31 | tableting adjunct | 1201 |
| CBD | 20.5 | 1.58 | essential element | |
| sesame oil | 5 | 0.38 | additive | 432 |
| PEG₃₀₀₀ | 10 | 0.77 | solubilizing excipient | 1521 |
| vitamin E TPGS | 25 | 1.92 | granulation agent | |
| mannitol 200 | 190 | 14.62 | bulking agent, | 421 |
| | | | sweetener | |
| microcrystalline cellulose | 37 | 2.85 | dispersing agent | 460(i) |
| (Avicel 102) | | | | |
| ascorbic acid (vitamin C) | 12 | 0.92 | antioxidant | 300 |
| tricalcium phosphate | 31.5 | 2.42 | anticaking agent | 341(iii) |
| silicon dioxide (Aerosil ® 200) | 15 | 1.15 | anticaking agent | 551 |
| magnesium stearate | 12 | 0.92 | anticaking agent | 470(iii) |
| butylated hydroxytoluene | 12 | 0.92 | antioxidant | 321 |
| total | 1300 | 100 | | |

TABLE 2
An anti-oxidant composition (tablet)

| | Amount | Percentage | | |
|---|---|---|---|---|
| Ingredient | (mg) | (%) | Function | INS |
| Mushroom mix: Flammulina | 900 | 69.23 | filler and functional | |
| velutipes CBS 146995 (25%), | | | excipient/ingredient | |
| Ganoderma lucidum CBS 146996 | | | | |
| (25%), Lentinus edodes (25%), | | | | |
| Trametes versicolor CBS 147000 | | | | |
| (25%) | | | | |
| polyvinylpyrrolidone K30 | 30 | 2.31 | tableting adjunct | 1201 |
| CBD | 20.5 | 1.58 | essential element | |
| sesame oil | 5 | 0.38 | additive | 432 |
| PEG₃₀₀₀ | 10 | 0.77 | solubilizing excipient | 1521 |
| vitamin E TPGS | 25 | 1.92 | granulation agent | |
| mannitol 200 | 190 | 14.62 | bulking agent, | 421 |
| | | | sweetener | |
| microcrystalline cellulose | 37 | 2.85 | dispersing agent | 460(i) |
| (Avicel 102) | | | | |
| ascorbic acid (vitamin C) | 12 | 0.92 | antioxidant | 300 |
| tricalcium phosphate | 31.5 | 2.42 | anticaking agent | 341(iii) |
| silicon dioxide (Aerosil ® 200) | 15 | 1.15 | anticaking agent | 551 |
| magnesium stearate | 12 | 0.92 | anticaking agent | 470(iii) |
| butylated hydroxytoluene | 12 | 0.92 | antioxidant | 321 |
| total | 1300 | 100 | | |

TABLE 3
An anti-psoriasis composition (tablet)

| | Amount | Percentage | | |
|---|---|---|---|---|
| Ingredient | (mg) | (%) | Function | INS |
| Mushroom mix: Inonotus obliquus | 900 | 69.23 | filler and functional | |
| (50%), Lentinus edodes (25%), | | | excipient/ingredient | |
| Ganoderma lucidum CBS 146996 | | | | |
| (25%) | | | | |
| polyvinylpyrrolidone K30 | 30 | 2.31 | tableting adjunct | 1201 |
| CBD | 20.5 | 1.58 | essential element | |
| sesame oil | 5 | 0.38 | additive | 432 |
| PEG₃₀₀₀ | 10 | 0.77 | solubilizing excipient | 1521 |
| vitamin E TPGS | 25 | 1.92 | granulation agent | |
| mannitol 200 | 190 | 14.62 | bulking agent, | 421 |
| | | | sweetener | |
| microcrystalline cellulose | 37 | 2.85 | dispersing agent | 460(i) |
| (Avicel 102) | | | | |
| ascorbic acid (vitamin C) | 12 | 0.92 | antioxidant | 300 |
| tricalcium phosphate | 31.5 | 2.42 | anticaking agent | 341(iii) |
| silicon dioxide (Aerosil ® 200) | 15 | 1.15 | anticaking agent | 551 |
| magnesium stearate | 12 | 0.92 | anticaking agent | 470(iii) |
| butylated hydroxytoluene | 12 | 0.92 | antioxidant | 321 |
| total | 1300 | 100 | | |

TABLE 4
A pain killer composition (tablet)

| | Amount | Percentage | | |
|---|---|---|---|---|
| Ingredient | (mg) | (%) | Function | INS |
| Mushroom mix: Cordyceps | 839 | 65.06 | filler and functional | |
| militaris (25%), Ganoderma | | | excipient/ingredient | |
| lucidum CBS 146996 (25%), | | | | |
| Inonotus obliquus (50%) | | | | |
| polyvinylpyrrolidone K30 | 30 | 2.33 | tableting adjunct | 1201 |
| CBD | 20.5 | 1.59 | essential element | |
| sesame oil | 5 | 0.39 | additive | 432 |
| PEG₃₀₀₀ | 10 | 0.78 | solubilizing excipient | 1521 |
| vitamin E TPGS | 25 | 1.94 | granulation agent | |
| mannitol 200 | 190 | 14.73 | bulking agent, | 421 |
| | | | sweetener | |
| microcrystalline cellulose | 36.5 | 2.83 | dispersing agent | 460(i) |
| (Avicel 102) | | | | |
| magnesium oxide | 94.5 | 7.33 | pain killer | 530 |
| silicon dioxide (Aerosil ® 200) | 15 | 1.16 | anticaking agent | 551 |
| magnesium stearate | 12 | 0.93 | anticaking agent | 470(iii) |
| butylated hydroxytoluene | 12 | 0.93 | antioxidant | 321 |
| total | 1289.5 | 100 | | |

TABLE 5
An anti-fatty liver composition (tablet)

| | Amount | Percentage | | |
|---|---|---|---|---|
| Ingredient | (mg) | (%) | Function | INS |
| Mushroom mix: Agaricus brasiliensis | 900 | 69.9 | filler and functional | |
| (15%), Coprinus comatus CBS 146994 | | | excipient/ingredient | |
| (15%), Cordyceps militaris (15%), | | | | |
| Lentinus edodes (40%), Pleurotus | | | | |
| ostreatus CBS 146999 (15%) | | | | |
| polyvinylpyrrolidone K30 | 30 | 2.33 | tableting adjunct | 1201 |
| CBD | 20.5 | 1.59 | essential element | |
| sesame oil | 5 | 0.39 | additive | 432 |
| PEG₃₀₀₀ | 10 | 0.78 | solubilizing excipient | 1521 |
| vitamin E TPGS | 25 | 1.94 | granulation agent | |
| mannitol 200 | 192 | 14.91 | bulking agent, | 421 |
| | | | sweetener | |
| microcrystalline cellulose | 39 | 3.03 | dispersing agent | 460(i) |
| (Avicel 102) | | | | |
| choline | 1.5 | 0.12 | anti fatty liver | |
| tricalcium phosphate | 25.5 | 1.98 | anticaking agent | 341(iii) |
| silicon dioxide (Aerosil ® 200) | 15 | 1.17 | anticaking agent | 551 |
| magnesium stearate | 12 | 0.93 | anticaking agent | 470(iii) |
| butylated hydroxytoluene | 12 | 0.93 | antioxidant | 321 |
| total | 1287.5 | 100 | | |

TABLE 6
An anti-diabetic composition (tablet)

| | Amount | Percentage | | |
|---|---|---|---|---|
| Ingredient | (mg) | (%) | Function | INS |
| Mushroom mix: Agaricus | 900 | 69.77 | filler and functional | |
| brasiliensis (20%), Coprinus | | | excipient/ingredient | |
| comatus CBS 146994 (40%), | | | | |
| Ganoderma lucidum CBS 146996 | | | | |
| (20%), Tremella fuciformis (20%) | | | | |
| polyvinylpyrrolidone K30 | 30 | 2.33 | tableting adjunct | 1201 |
| CBD | 20.5 | 1.59 | essential element | |
| sesame oil | 5 | 0.39 | additive | 432 |
| PEG₃₀₀₀ | 10 | 0.78 | solubilizing excipient | 1521 |
| vitamin E TPGS | 25 | 1.94 | granulation agent | |
| mannitol 200 | 190 | 14.73 | bulking agent, | 421 |
| | | | sweetener | |
| microcrystalline cellulose | 37 | 2.87 | dispersing agent | 460(i) |
| (Avicel 102) | | | | |
| chromium picolinate | 0.01 | 0 | anti diabetic | |
| tricalcium phosphate | 33.5 | 2.6 | anticaking agent | 341(iii) |
| silicon dioxide (Aerosil ® 200) | 15 | 1.16 | anticaking agent | 551 |
| magnesium stearate | 12 | 0.93 | anticaking agent | 470(iii) |
| butylated hydroxytoluene | 12 | 0.93 | antioxidant | 321 |
| total | 1290.01 | 100 | | |

TABLE 7
A bone strengthener composition (tablet)

| | Amount | Percentage | | |
|---|---|---|---|---|
| Ingredient | (mg) | (%) | Function | INS |
| Mushroom mix: Cordyceps militaris | 772 | 59.71 | filler and functional | |
| (20%), Grifola frondosa CBS 146997 | | | excipient/ingredient | |
| (20%), Tremella fuciformis (20%), | | | | |
| Lentinus edodes (20%), Ganoderma | | | | |
| lucidum CBS 146996 (20%) | | | | |
| polyvinylpyrrolidone K30 | 30 | 2.32 | tableting adjunct | 1201 |
| CBD | 20.5 | 1.59 | essential element | |
| sesame oil | 5 | 0.39 | additive | 432 |
| PEG₃₀₀₀ | 10 | 0.77 | solubilizing excipient | 1521 |
| vitamin E TPGS | 25 | 1.93 | granulation agent | |
| mannitol 200 | 190 | 11.60 | bulking agent, sweetener | 421 |
| microcrystalline cellulose | 37 | 2.86 | dispersing agent | 460(i) |
| (Avicel 102) | | | | |
| ascorbic acid (vitamin C) | 12 | 0.93 | antioxidant | 300 |
| dicalcium phosphate | 192.5 | 14.89 | anticaking agent | 341(iii) |
| silicon dioxide (Aerosil ® 200) | 15 | 1.16 | anticaking agent | 551 |
| magnesium stearate | 12 | 0.93 | anticaking agent | 470(iii) |
| butylated hydroxytoluene | 12 | 0.93 | antioxidant | 321 |
| total | 1293 | 100 | | |

TABLE 8
An anti-arrhythmic composition (tablet)

| | Amount | Percentage | | |
|---|---|---|---|---|
| Ingredient | (mg) | (%) | Function | INS |
| Mushroom mix: Pleurotus ostreatus CBS | 900 | 69.82 | filler and functional | |
| 146999 (40%), Ganoderma lucidum CBS | | | excipient/ingredient | |
| 146996 (15%), Auricularia aricula-judae | | | | |
| (15%), Grifola frondosa CBS 146997 | | | | |
| (15%), Lentinus edodes (15%) | | | | |
| polyvinylpyrrolidone K30 | 30 | 2.33 | tableting adjunct | 1201 |
| CBD | 20.5 | 1.59 | essential element | |
| sesame oil | 5 | 0.39 | additive | 432 |
| PEG₃₀₀₀ | 10 | 0.78 | solubilizing excipient | 1521 |
| vitamin E TPGS | 25 | 1.94 | granulation agent | |
| mannitol 200 | 190 | 14.74 | bulking agent, | 421 |
| | | | sweetener | |
| microcrystalline cellulose (Avicel 102) | 37 | 2.87 | dispersing agent | 460(i) |
| vitamin B1 thiamin | 1 | 0.08 | anti arrytmic | |
| tricalcium phosphate | 31.5 | 2.44 | anticaking agent | 341(iii) |
| silicon dioxide (Aerosil ® 200) | 15 | 1.16 | anticaking agent | 551 |
| magnesium stearate | 12 | 0.93 | anticaking agent | 470(iii) |
| butylated hydroxytoluene | 12 | 0.93 | antioxidant | 321 |
| total | 1289 | 100 | | |

TABLE 9
An immunoguard composition (tablet)

| | Amount | Percentage | | |
|---|---|---|---|---|
| Ingredient | (mg) | (%) | Function | INS* |
| Mushrooms mix: Trametes | 900 | 66.32 | filler and functional | |
| versicolor CBS 147000 (20%), | | | excipient/ingredient | |
| Cordyceps militaris (20%), | | | | |
| Flammulina velutipes CBS | | | | |
| 146995 (20%), Ganoderma | | | | |
| lucidum CBS 146996 (20%), | | | | |
| Lentinus edodes (20%) | | | | |
| Ganoderma lucidum alcoholic | 100 | 7.37 | | |
| extract (GLAE) powder | | | | |
| (Shanghai Sanmin Herbal | | | | |
| Industrial Co., Ltd.) | | | | |
| polyvinylpyrrolidone K30 | 30 | 2.21 | tableting adjunct | 1201 |
| CBD | 15.3 | 1.13 | essential element | |
| sesame oil | 5 | 0.37 | additive | 432 |
| PEG₃₀₀₀ | 10 | 0.74 | solubilizing excipient | 1521 |
| vitamin E TPGS | 25 | 1.84 | granulation agent | |
| mannitol 200 | 118.4 | 8.73 | bulking agent, | 421 |
| | | | sweetener | |
| microcrystalline cellulose | 37 | 2.73 | dispersing agent | 460(i) |
| (Avicel 102) | | | | |
| zinc citrate | 43.8 | 3.23 | immunoguard | |
| tricalcium phosphate | 33.5 | 2.47 | anticaking agent | 341(iii) |
| silicon dioxide (Aerosil ® 200) | 15 | 1.11 | anticaking agent | 551 |
| magnesium stearate | 12 | 0.88 | anticaking agent | 470(iii) |
| butylated hydroxytoluene | 12 | 0.88 | antioxidant | 321 |
| total | 1357 | 100 | | |

### Example 2. Stability of the CBD-Mushroom-Based Nutraceutical Composition Prepared

### Materials and Methods

HPLC analysis was performed using LiChrosphero 60 RP-select B (5 μm) 250×4 mm column at 30° C.; acetonitrile:water (80:20 v/v) as the solvent system, at a flow rate of 1 mL/min and total run time of 10 min; and 10 μL injection volume. The retention time of CBD under these conditions was 4-6 min.

The various tablets described in Tables 1-9 were stored at 40±2° C./75±5% relative humidity (RH), and their stability for up to 6 months was evaluated using HPLC (see Table 10).

TABLE 10
stability data of the tablets described in Tables 1-9

| | | Tests and specifications | | | | | |
|---|---|---|---|---|---|---|---|
| | | | | | Total | | |
| | | | | | impurities/ | | |
| | Testing | | Assay | | degradation | Dissolution | Water |
| | interval | | (HPLC) | | products | rate (0.1 M | content |
| Indication | (month) | Appearance | mg/tab (%) | | (HPLC; %) | SLS; %/hour) | (%) |
| Specifications | | green coated | 20.0 ± 2.0 | | NMT 6.0% | NLT 75 | NMT 10 |
| | | oval tablet | (90.0-110.0%) | | | | |
| antiaging | initial | conform | 19.7 | (98.5%) | 0% | 93.5 | 5.7 |
| | 3 M | conform | 19.8 | (99%) | 0% | 101.4 | 5.8 |
| | 6 M | conform | 19.0 | (95%) | 0% | 99.9 | 4.7 |
| antioxidant | initial | conform | 19.9 | (99.5%) | 0% | 93.0 | 5.9 |
| | 3 M | conform | 20.0 | (100%) | 0% | 99.7 | 4.1 |
| | 6 M | conform | 19.2 | (96%) | 0% | 98.7 | 4.9 |
| anti-psoriasis | initial | conform | 19.8 | (99%) | 0% | 92.7 | 5.8 |
| | 3 M | conform | 19.1 | (95.5%) | 0% | 98.6 | 5.3 |
| | 6 M | conform | 18.6 | (93%) | 0% | 100.8 | 5.4 |
| bone | initial | conform | 20.1 | (100.5%) | 0% | 82.1 | 5.8 |
| strengthener | 3 M | conform | 19.4 | (97%) | 0% | 94.6 | 3.3 |
| | 6 M | conform | 19.0 | (95%) | 0% | 93.8 | 4.5 |
| anti-arrhythmic | initial | conform | 20.0 | (100%) | 0% | 92.7 | 5.5 |
| | 3 M | conform | 18.8 | (94%) | 0% | 99.2 | 4.0 |
| | 6 M | conform | 18.5 | (92.5%) | 0% | 100.8 | 4.0 |
| pain killer | Initial | conform | 20.2 | (101%) | 0% | 44.6 | 6.0 |
| | 3 M | conform | 20.1 | (100.5%) | 0% | 20.8 | 4.8 |
| | 6 M | conform | 20.3 | (101.5%) | 0% | NA | 4.0 |
| immunoguard | initial | conform | 15.1 | (100.6%) | 0% | 87.8 | 4.8 |
| | 3 M | conform | 14.7 | (98%) | 0% | 93.6 | 5.4 |
| | 6 M | conform | 14.4 | (96%) | 0% | 91.7 | 4.7 |
| Anti-diabetes | initial | conform | 18.8 | (94%) | 0% | NA | NA |
| | 3 M | conform | 17.4 | (87%) | 0% | 100.1 | 4.2 |
| | 6 M | conform | 16.7 | (83.5%) | 0% | 100.9 | 2.9 |
| Anti-fatty liver | initial | conform | 20.0 | (100%) | 0% | 92.9 | 6.5 |
| | 3 M | conform | 19.7 | (98.5%) | 0% | 100.1 | 4.6 |
| | 6 M | conform | 19.4 | (97%) | 0% | 97.9 | 4.7 |

BRL—below reporting level (0.1%)
ND—not detected
NMT—not more than
NLT—not less than
NA—not analyzed
SLS—sodium lauryl sulfate (simulating gastrointestinal conditions in vitro)

### Results and Conclusions

The tablets were found to be stable at accelerating conditions of 40±2° C./75±5% RH for 6 months, corresponding to 2 years at normal ambient conditions, except for the following cases: (i) due to the very low dissolution rate of the pain killer tablets at 3 months stability point, this tablet was not analyzed for the dissolution rate at 6 months stability point; and (ii) the assay of the anti-diabetic tablets decreased for 9.5% within 6 months, and its test results for 3 months (87%) and 6 months (83.5%) are out-of-specifications.

### Example 3. Dissolution Profile of CBD-Mushroom-Based Nutraceutical Composition

The dissolution profile of CBD-mushroom-based nutraceutical compositions as disclosed herein, more specifically the composition referred herein to as “CBD-176-66-TB” (content summarized in Table 11), prepared by wet granulation as described above, was performed under fast conditions, using Fasted State Simulating Intestinal Fluids (FaSSIF) media (there are no specific USP/EP methods for evaluating the dissolution profile of CBD-containing products). These media contain natural surfactants (bile salts and phospholipids) and thus simulate the gastrointestinal fluids much more accurately than conventional dissolution media, further taking into account the absence/presence of food (drugs in Fasted State media, i.e., before a meal, often behave differently from those in the Fed State, i.e., after a meal). These media are useful when developing a drug product for oral administration, as they may help predicting how the drug is likely to perform in the gut.

TABLE 11
Content of the CBD-176-66-TB tablets

| | Cores | | liquids | Per 68 tablets |
|---|---|---|---|---|
| ingredient | %/tablet | mg/tablet | mg/caplet | (gr) |

Part I - wet granulation

| | | | | |
|---|---|---|---|---|
| Mushroom mix: Pleurotus ostreatus | 71.15 | 900 | | 61.20 |
| CBS 146999 (30%), Ganoderma | | | | |
| lucidum CBS 146996 (20%), | | | | |
| Auricularia aricula-judae (20%), | | | | |
| Grifola frondosa CBS 146997 (15%), | | | | |
| Lentinus edodes (15%) | | | | |
| CBD | 1.98 | 25 | | 1.70 |
| Kollidon ® 30 (a polyvinylpyrrolidone | 2.37 | 30 | | 2.04 |
| polymer) | | | | |
| BHT | 0.79 | 10 | | 0.68 |
| PEG₃₀₀₀ | 0.4 | 5 | | 0.34 |
| sesame oil | 0.4 | 5 | | 0.34 |
| vitamin E TPGS | 1.98 | 25 | | 1.70 |
| ethanol | | | 180 | 12.24 |

Part II - direct compression

| | | | | |
|---|---|---|---|---|
| mannitol 200 | 15.02 | 190 | | 12.92 |
| microcrystalline cellulose (Avicel 102) | 2.77 | 35 | | 2.38 |
| silicon dioxide (Aerosil ® 200) | 1.58 | 20 | | 1.36 |
| magnesium stearate | 1.58 | 20 | | 1.36 |
| total | 100 | 1265 | 180 | 86 |

### Materials and Methods

Equipment. Balance; Bin 1L; Dynamic Tablet press; Hardness tester TH3; Balance; Bin 1L; Dynamic Tablet press.

Preparation steps. The CBD tablets were prepared following steps 1-6 hereinbelow:

Step 1. The mushroom mix was loaded into a Diosna wet granulation machine (0.5 L) and was mixed for 1 minute with impeller rate of 300 rpm and chopper rate of 500 rpm, prior to the addition of the solution from step 2.

Step 2. Vitamin E TPGS, Kollidon® 30, PEG₃₀₀₀, BHT, sesame oil, ethanol, and CBD were mixed with a magnetic stirrer at a temperature of 30° C. for about 20 minutes in a separate vessel, until a yellow clear solution was obtained.

Step 3. The solution obtained in step 2 was added to the mushroom mixture obtained in step 1 and mixing continued for 1-3 minute until homogeneity was achieved, with impeller rate of 500 rpm and chopper rate 700 rpm, to obtain a wet granulation premix.

Step 4. The obtained wet mass was transferred into a vacuum oven and dried in a temperature of about 35-45° C. for about 60 min until the ethanol was evaporated. The dried granules obtained were milled manually by passing through a 1400 microns screen sieve, and then through 850 microns net.

Step 5. The materials from part II were sieved through a 850-micron screen sieve, and then transferred into the mixer bin (0.5 L) and mixed for 15 min at 10 rpm rate. Then, 1% Mg stearate was added, and mixing continued for 3 min.

Step 6. The obtained mixture was compressed into tablets using a tablet machine (Dynamic).

### Results

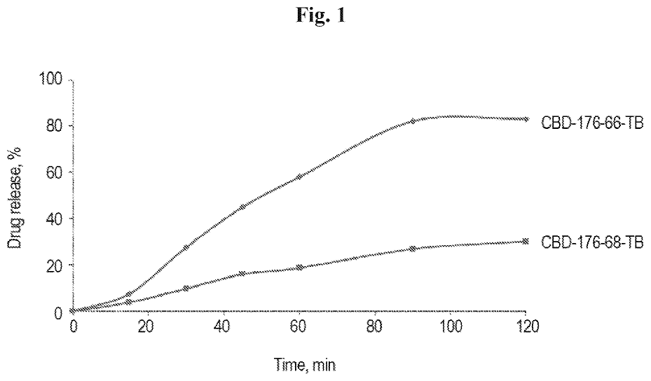

The solubility of the tablets prepared vs. control tablets (referred to herein as CBD-176-68-TB) comprising the same amount (25 mg) of crystalline but unprocessed CBD was observed in FaSSIF. The results are shown in FIG. 1 and summarized in Table 12. As shown, the wet granulated delivery matrix system (based on vitamin E TPGS) significantly improved CBD solubility, and probably bioavailability, in the caplet product CBD-176-66-TB, wherein three folds more CBD (82%) was released from those tablets compared to the control tablets.

TABLE 12
CBD dissolution from improved formulation coated tablet (CBD-
176-66-TB) and control tablet (CBD-176-68-TB) in FaSSIF media

| | |
|---|---|
| Time | Percentage of CBD released (%) |

| (min) | CBD-176-66-TB | CBD-176-68-TB |
|---|---|---|
| 0 | 0.0 | 0.0 |
| 15 | 7.5 | 3.8 |
| 30 | 27.5 | 9.7 |
| 45 | 44.9 | 16.0 |
| 60 | 57.9 | 18.7 |
| 90 | 82.0 | 26.9 |
| 120 | 83.0 | 30.2 |

### Example 4. Bioavailability of CBD from CBD-Mushroom-Based Nutraceutical Composition in Animal Model

Pharmacokinetic evaluation of CBD in rats. This study is aimed at determining the pharmacokinetic profile of CBD following single administration of CBD-based tablets as depicted in Table 11 as compared to CBD granules prepared from crystalline CBD without delivery system and tableting excipients (referred to as “control”). In both cases, administration is done to male Wistar rats (275-300 g) by oral gavage, at a dosing of 50 mg/kg (10 mg per rat) (higher dosage of, e.g., up to 100 mg/kg, may be administered as well).

Body weight of the rats is measured during acclimation and before the administration of CBD (either as the control or the CBD tablets). During all the experiment, toxic signs after dosing, as well as morbidity and mortality of the rats, are monitored daily.

Blood is collected 24 hours before dosing (baseline), and every 30 minutes after administration, for a period of 12 hours (at each time point, blood is collected from three rats), and the plasma concentrations of both CBD and its metabolites are measured by using a high-pressure liquid chromatograph-tandem mass spectrometer system (LC-MS/MS), so as to have the Tₘₐₓ, Cₘₐₓ and AUCₜ (area under the concentration-time curve from zero up to a definite time t; This parameter refers to the plasma drug level at each time point and thus used as an index of the drug exposure to the body, and is closely dependent on the drug amount that enter into the systemic circulation). Superior absorption (statistically significant) of CBD is expected from the tested formulation compared to that from the control formulation.

### REFERENCES

Amidon G. L., Lennerniis H., Shah V. P., Crison, J. R. A theoretical basis for a biopharmaceutic drug classification: the correlation of in vitro drug product dissolution and in vivo bioavailability. Pharmaceutical research, 1995, 12, 413-420
Badalyan S. M., Singh M. Potential of mushroom bioactive molecules to develop healthcare biotech products. in Proceedings of the 8th International Conference on mushroom biology and mushroom products (ICMBMP8) (pp. 373-378). New Delhi: Yugantar Prakashan Pvt. Ltd., 2014
Beulah G. H., Margret A. A., Nelson J. Marvelous medicinal mushrooms, 2013
Bittner S. When quinones meet amino acids: chemical, physical and biological consequences. Amino acids, 2006, 30, 205-224
Dressman J. B., Reppas C. In vitro—in vivo correlations for lipophilic, poorly water-soluble drugs. European journal of pharmaceutical sciences, 2000, 11, S73-S80
Fleisher D., Li C., Zhou Y., Pao L. H., Karim A. Drug, meal and formulation interactions influencing drug absorption after oral administration. Clinical pharmacokinetics, 1999, 36, 233-254
Giavasis 1. Bioactive fungal polysaccharides as potential functional ingredients in food and nutraceuticals. Current opinion in biotechnology, 2014a, 26, 162-173
Giavasis I. Polysaccharides from medicinal mushrooms for potential use as nutraceuticals. Polysaccharides: Natural Fibers in Food and Nutrition, 2014b, 1, 171-206
Kostewicz E. S., Brauns U., Becker R., Dressman J. B. Forecasting the oral absorption behavior of poorly soluble weak bases using solubility and dissolution studies in biorelevant media. Pharmaceutical research, 2002, 19, 345
Kothari D., Patel S., Kim S. K. Anticancer and other therapeutic relevance of mushroom polysaccharides: A holistic appraisal. Biomedicine & Pharmacotherapy, 2018, 105, 377-394
Lennernaäs H. Human intestinal permeability. Journal of pharmaceutical sciences, 1998, 87, 403-410
Lennernäs H., Abrahnamsson B. The use of biophannaceutic classification of drugs in drug discovery and development: current status and future extension. Journal of pharmacy and pharmacology, 2005, 57, 273-285
Marques M. Dissolution media simulating fasted and fed states. Dissolution Technologies, 2004, 11, 16-19
Pardeshi B., Pardeshi P. The edible medicinal mushrooms as supportive natural nutrients: Study of nonvolatile mineral contents of some edible medicinal mushrooms from India: eastern remedies for modern western maladies. In Proceedings of the 5˜(th) international medicinal mushroom conference, 2009
Pereira E., Barros L., Martins A., Ferreira I. C. Towards chemical and nutritional inventory of Portuguese wild edible mushrooms in different habitats. Food Chemistry, 2012, 130, 394-403
Polli J. E., Lawrence X. Y., Cook J. A., Amidon G. L., Borchardt R. T., Burnside B. A., Burton P. S, Chen M. L., Conner D. P., Faustino P. J., Hawi A. A., Hussain A. S., Joshi H. N., Kwei G., Lee V. H. L., Lesko L. J., Lipper R. A., Loper A. E., Nerurkar S. G., Polli J. W., Sanvordeker D. R., Taneja R., Uppoor R. S., Vattikonda C. S., Wilding L., Zhang, G. Summary workshop report: biopharmaceutics classification system—implementation challenges and extension opportunities. Journal of pharmaceutical sciences, 2004, 93, 1375-1381
Rathee S., Rathee D., Rathee D., Kumar V., Rathee P. Mushrooms as therapeutic agents. Revista Brasileira de Farnacognosia, 2012, 22, 459-474.
Roupas P., Keogh J., Noakes M., Margetts C., Taylor P. The role of edible mushrooms in health: Evaluation of the evidence. Journal of Functional Foods, 2012, 4, 687-709
Ruiz-Rodriguez A., Santoyo S., Soler-Rivas, C. Antioxidant properties of edible mushrooms. Funct Plant Sci Biotechnol, 2009, 3, 92-102
Singh R. A review on different benefits of mushroom. IOSR Journal of Pharmacy and Biological Sciences, 2017, 12, 107-11
Üstün N. S., Bulam S., Pekçen A. The use of mushrooms and their extracts and compounds in functional foods and nutraceuticals. Tûrkmen, A.(ed.), 2018, 1, 1205-1222
Vaz J. A., Heleno S. A., Martins A., Almeida G. M., Vasconcelos M. H., Ferreira I. C. Wild mushrooms Clitocybe alexandri and Lepista inversa: in vitro antioxidant activity and growth inhibition of human tumour cell lines. Food and Chemical Toxicology, 2010, 48, 2881-2884
Wasser S. P., Weis A. L. Medicinal properties of substances occurring in higher basidiomycetes mushrooms: current perspectives. International Journal of medicinal mushrooms, 1999a, 1, 31-62
Wasser S. P., Weis A. L. Therapeutic effects of substances occurring in higher Basidiomycetes mushrooms: A modem perspective. Critical Reviews in Immunology, 1999b, 19, 65-96
Wasser S. Medicinal mushroom science: Current perspectives, advances, evidences, and challenges. Biomedical journal, 2014, 37, 345-356
Yokota M. E., Frison P. S., Marcante R. C., Jorge L. F., Valle J. S., Dragunski D. C., Colauto N. B., Linde G. A. Iron translocation in Pleurotus ostreatus basidiocarps: production, bioavailability, and antioxidant activity. Genetics and Molecular Research, 2016
Yu L X., Amidon G. L., Polli J. E., Zhao H., Mehta M. U., Conner D. P., Shah V. P., Lesko L. J., Chen M. L., Lee V. H. L., Hussain A. S. Biopharmaceutics classification system: the scientific basis for biowaiver extensions. Pharmaceutical research, 2002, 19, 921-925

1-30. (canceled)

31. A composition comprising a cannabinoid as an active agent, D-α-tocopherol polyethylene glycol 1000 succinate (TPGS), and an excipient selected from the group consisting of polyethylene glycol (PEG) having a molecular weight of from about 2 kDa to about 50 kDa, and polyvinylpyrrolidone (PVP).

32. The composition of claim 31, wherein said cannabinoid is cannabidiol (CBD), cannabidiolic acid (CBDA), cannabidiphorol (CBDP), cannabidivarin (CBDV), cannabidivarinic acid (CBDVA), cannabidiol monomethyl ether (CBDM), cannabidiol-C4 (CBD-C4), cannabidiorcol (CBD-C1), Δ⁹-tetrahydrocannabinol (Δ⁹-THC), Δ⁹-tetrahydrocannabinolic acid (Δ⁹-THCA), Δ⁹-tetrahydrocannabivarin (Δ⁹-THCV), Δ⁹-tetrahydrocannabivarinic acid (Δ⁹-THCVA), Δ⁸-THC, Δ⁸-THCA, Δ⁸-THCV, Δ⁸-THCVA, iso-tetrahydrocannabinol-type (iso-THC), cannabinol (CBN), cannabinolic acid (CBNA), cannabinol-C4 (CBN-C4), cannabinol-C2 (CBN-C2), cannabiorcol (CBN-C1), cannabinol methyl ether (CBNM), cannabinodiol (CBND), cannabigerol (CBG), cannabigerovarin (CBGV), cannabigerolic acid (CBGA), cannabigerovarinic acid (CBGVA), cannabigerol monomethyl ether (CBGM), cannabigerolic acid monomethyl ether (CBGAM), cannabichromene (CBC), cannabichromenic acid (CBCA), cannabichromevarin (CBCV), cannabichromevarinic acid (CBCVA), cannabichromanon (CBCN), cannabicyclol (CBL), cannabicyclolic acid (CBLA), cannabicyclovarin (CBLV), cannabivarin (CBV), cannabivarinic acid (CBVA), cannabielsoin (CBE), cannabielsoic acid A (CBEA-A), cannabielsoic acid B (CBEA-B), cannabitriol (CBT), cannabitriolvarin (CBTV), ethoxy-cannabitiolvarin (CBTVE), cannabifuran (CBF), dehydrocannabifuran (DCBF), cannabiripsol (CBR), an enantiomer, diastereomer, or salt thereof, or a mixture thereof.

33. The composition of claim 32, wherein said cannabinoid is CBD or an enantiomer, diastereomer, or mixture thereof.

34. The composition of claim 31, wherein the ratio between said cannabinoid and said TPGS is from about 50:1 to about 1:50 by weight, respectively; and the ratio between said cannabinoid and said excipient is from about 1:0.1 to about 1:1 by weight, respectively.

35. The composition of claim 31, further comprising an antioxidant.

36. The composition of claim 35, wherein the ratio between said cannabinoid and said antioxidant is from about 1:0.1 to about 1:10 by weight, respectively.

37. The composition of claim 31, wherein said cannabinoid is CBD or an enantiomer, diastereomer, or mixture thereof; said excipient is PEG; the ratio between said CBD and said TPGS is from about 10:1 to about 1:10 by weight, respectively; and the ratio between said CBD and said PEG is from about 1:0.4 to about 1:0.8 by weight, respectively.

38. The composition of claim 58, wherein said antioxidant is butylated hydroxytoluene (BHT), and the ratio between said CBD and said BHT is from about 1:0.8 to about 1:2 by weight, respectively.

39. The composition of claim 31, further comprising a pharmaceutically acceptable carrier.

40. The composition of claim 31, further comprising a nutraceutically acceptable carrier.

41. The composition of claim 40, further comprising at least one additional active agent, and optionally a binder.

42. The composition of claim 41, wherein said binder is a saccharide, a polysaccharide, a natural binder, a synthetic or semi-synthetic polymer, or a mixture thereof.

43. The composition of claim 41, which comprises said binder, wherein said at least one additional active agent is a biomass of, or an extract obtained from, a higher Basidiomycetes mushroom or a combination thereof.

44. The composition of claim 43, wherein said additional active agent is an extract obtained from a higher Basidiomycetes mushroom or a combination thereof, and at least one of said higher Basidiomycetes mushrooms is selected from the group consisting of:
(i) Coprinus comatus (O. F. Mill.) Pers. deposited under the Budapest Treaty with the Centraalbureau voor Schimmelcultures (CBS) under Accession No. CBS 146994 (hereinafter Coprinus comatus CBS 146994);
(ii) Flammulina velutipes (Curtis) Singer deposited under the Budapest Treaty with the CBS under Accession No. CBS 146995 (hereinafter Flammulina velutipes CBS 146995);
(iii) Ganoderma lucidum (Curtis: Fr.) P. Karst. deposited under the Budapest Treaty with the CBS under Accession No. CBS 146996 (hereinafter Ganoderma lucidum CBS 146996);
(iv) Grifola frondosa (Dicks.) Gray deposited under the Budapest Treaty with the CBS under Accession No. CBS 146997 (hereinafter Grifola frondosa CBS 146997);
(v) Hericium erinaceus (Bull.: Fr.) Pers. deposited under the Budapest Treaty with the CBS under Accession No. CBS 146998 (hereinafter Hericium erinaceus CBS 146998);
(vi) Pleurotus ostreatus (Jacq.: Fr.) Kummer deposited under the Budapest Treaty with the CBS under Accession No. CBS 146999 (hereinafter Pleurotus ostreatus CBS 146999); and
(vii) Trametes versicolor (L.: Fr.) Lloyd. deposited under the Budapest Treaty with the CBS under Accession No. CBS 147000 (hereinafter Trametes versicolor CBS 147000).

45. The composition of claim 44, wherein at least another one of said higher Basidiomycetes mushrooms is selected from the group consisting of Agaricus brasiliensis, Auricularia aricula-judae, Cordyceps militaris, Ganoderma tsugae, Hypsizygus marmoreus, Inonotus obliquus, Lentinus edodes, Pleurotus eryngii, and Tremella fuciformis.

46. The composition of claim 43, wherein said extract is obtained by extracting said higher Basidiomycetes mushroom or combination thereof with water at a temperature of about 60-90° C.

47. The composition of claim 60, wherein said cannabinoid constitutes from about 0.5% to about 3% by weight of said composition; said excipient constitutes from about 0.3% to about 1.5% by weight of said composition; said binder constitutes from about 1.5% to about 4% by weight of said composition; and said at least one additional agent constitutes from about 50% to about 80% by weight of said composition.

48. The composition of claim 47, further comprising an antioxidant.

49. The composition of claim 48, wherein said antioxidant constitutes from about 0.5% to about 3% by weight of said composition.

50. composition of claim 47, wherein said at least one additional agent is an extract obtained from a higher Basidiomycetes mushrooms or a combination thereof.

51. The composition of claim 50, wherein said antioxidant is selected from the group consisting of BHT, ascorbic acid, and a mixture thereof; the amount of said PEG in said composition is from about 0.5% to about 1.5% by weight; and the amount of said antioxidant in said composition is from about 0.7% to about 1.5% by weight.

52. The composition of claim 61, comprising:
(i) CBD in an amount of about 1.6% by weight; TPGS in an amount of about 1.9% by weight; PEG having a molecular weight of about 3 kDa, in an amount of about 0.8% by weight; a mixture of BHT and ascorbic acid each independently in an amount of about 0.9% by weight; and an extract obtained from a combination of the higher Basidiomycetes mushrooms Coprinus comatus CBS 146994, Lentinus edodes, and Tremella fuciformis, in an amount of about 69% by weight;
(ii) CBD in an amount of about 1.6% by weight; TPGS in an amount of about 1.9% by weight; PEG having a molecular weight of about 3 kDa, in an amount of about 0.8% by weight; a mixture of BHT and ascorbic acid each independently in an amount of about 0.9% by weight; and an extract obtained from a combination of the higher Basidiomycetes mushrooms Flammulina velutipes CBS 146995, Ganoderma lucidum CBS 146996, Lentinus edodes, and Trametes versicolor CBS 147000, in an amount of about 69% by weight;
(iii) CBD in an amount of about 1.6% by weight; TPGS in an amount of about 1.9% by weight; PEG having a molecular weight of about 3 kDa, in an amount of about 0.8% by weight; a mixture of BHT and ascorbic acid each independently in an amount of about 0.9% by weight; and an extract obtained from a combination of the higher Basidiomycetes mushrooms Inonotus obliquus, Lentinus edodes, and Ganoderma lucidum CBS 146996, in an amount of about 69% by weight);
(iv) CBD in an amount of about 1.6% by weight; TPGS in an amount of about 1.9% by weight; PEG having a molecular weight of about 3 kDa, in an amount of about 0.8% by weight; BHT in an amount of about 0.9% by weight; and an extract obtained from a combination of the higher Basidiomycetes mushrooms Cordyceps militaris, Ganoderma lucidum CBS 146996, and Inonotus obliquus, in an amount of about 65% by weight;
(v) CBD in an amount of about 1.6% by weight; TPGS in an amount of about 1.9% by weight; PEG having a molecular weight of about 3 kDa, in an amount of about 0.8% by weight; BHT in an amount of about 0.9% by weight; and an extract obtained from a combination of the higher Basidiomycetes mushrooms Agaricus brasiliensis, Coprinus comatus CBS 146994, Cordyceps militaris, Lentinus edodes, and Pleurotus ostreatus CBS 146999, in an amount of about 70% by weight;
(vi) CBD in an amount of about 1.6% by weight; TPGS in an amount of about 1.9% by weight; PEG having a molecular weight of about 3 kDa, in an amount of about 0.8% by weight; BHT in an amount of about 0.9% by weight; and an extract obtained from a combination of the higher Basidiomycetes mushrooms Agaricus brasiliensis, Coprinus comatus CBS 146994, Ganoderma lucidum CBS 146996, and Tremella fuciformis, in an amount of about 70% by weight;
(vii) CBD in an amount of about 1.6% by weight; TPGS in an amount of about 1.9% by weight; PEG having a molecular weight of about 3 kDa, in an amount of about 0.8% by weight; a mixture of BHT and ascorbic acid each independently in an amount of about 0.9% by weight; and an extract obtained from a combination of the higher Basidiomycetes mushrooms Cordyceps militaris, Grifola frondosa CBS 146997, Tremella fuciformis, Lentinus edodes, and Ganoderma lucidum CBS 146996, in an amount of about 60% by weight;
(viii) CBD in an amount of about 1.6% by weight; TPGS in an amount of about 2% by weight; PEG having a molecular weight of about 3 kDa, in an amount of about 0.8% by weight; BHT in an amount of about 0.9% by weight; and an extract obtained from a combination of the higher Basidiomycetes mushrooms Pleurotus ostreatus CBS 146999, Ganoderma lucidum CBS 146996, Auricularia aricula-judae, Grifola frondosa CBS 146997, and Lentinus edodes, in an amount of about 70% by weight; or
(ix) CBD in an amount of about 1.2% by weight; TPGS in an amount of about 1.8% by weight; PEG having a molecular weight of about 3 kDa, in an amount of about 0.8% by weight; BHT in an amount of about 0.9% by weight; and an extract obtained from a combination of the higher Basidiomycetes mushrooms Trametes versicolor CBS 147000, Cordyceps militaris, Flammulina velutipes CBS 146995, Ganoderma lucidum CBS 146996, and Lentinus edodes, in an amount of about 66% by weight.

53. The composition of claim 31, in the form of a liquid, semi-solid, or solid.

54. The composition of claim 53, formulated as a solution in an edible solvent or a solid dosage form.

55. A drink, beverage, or food supplement, comprising a composition according to claim 40.

56. The composition of claim 31, wherein said excipient is PEG having a molecular weight of from about 2 kDa to about 50 kDa.

57. The composition of claim 35, wherein said antioxidant is selected from the group consisting of butylated hydroxytoluene (BHT); ascorbic acid (vitamin C) or a stereoisomer thereof; a tocopherol or a derivative thereof; 4-hexylresorcinol; propyl gallate; butylated hydroxyanisole (BHA); rosemary extract; and a mixture thereof.

58. The composition of claim 37, further comprising an antioxidant.

59. The composition of claim 42, wherein said saccharide is sucrose or glucose; said polysaccharide is starch or cellulose; said natural binder is acacia (gum Arabic), tragacanth, gelatin, or alginic acid; or said synthetic or semi-synthetic polymer is a cellulose-based polymer, polyvinyl pyrrolidone (PVP), polyvinyl alcohol, PEG, or polymethacrylate.

60. The composition of claim 43, wherein said cannabinoid is CBD or an enantiomer, diastereomer, or mixture thereof; and said excipient is PEG having a molecular weight of from about 2 kDa to about 50 kDa.

61. The composition of claim 50, wherein at least one of said higher Basidiomycetes mushrooms is selected from the group consisting of hereinafter Coprinus comatus CBS 146994, Flammulina velutipes CBS 146995, Ganoderma lucidum CBS 146996, Grifola frondosa CBS 146997, Hericium erinaceus CBS 146998, Pleurotus ostreatus CBS 146999, and Trametes versicolor CBS 147000; at least another one of said higher Basidiomycetes mushrooms is selected from the group consisting of Agaricus brasiliensis, Auricularia aricula-judae, Cordyceps militaris, Ganoderma tsugae, Hypsizygus marmoreus, Inonotus obliquus, Lentinus edodes, Pleurotus eryngii, and Tremella fuciformis; and wherein said extract is obtained by extracting said combination of higher Basidiomycetes mushrooms with water at a temperature of about 60-90° C.
